(19) United States
(12) Patent Application Publication
(10) Pub. No.: US 2014/0323444 A1
(43) Pub. Date: Oct. 30, 2014

(54) ANTI-INFLAMMATORY AND ANTIDIABETIC AGENTS

(71) Applicant: Banavara L. Mylari, Lutz, FL (US)

(72) Inventor: Banavara L. Mylari, Lutz, FL (US)

(21) Appl. No.: 13/869,117

(22) Filed: Apr. 24, 2013

(51) Int. Cl. A61K 31/616

(52) U.S. Cl. CPC A61K 31/616
USPC 514/160

(57) ABSTRACT

The present invention relates to compounds,

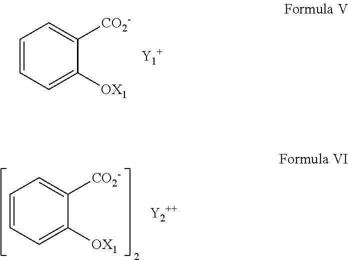

intermediates used in the preparation of such compounds, processes for the preparation of such compounds of the formula V and formula VI and such intermediates, pharmaceutical compositions comprising such compounds of the formula V and such compounds of the formula VI, and the uses of such compounds of the formula V and such compounds of the formula VI as anti-inflammatory, antiobesity and cardioprotective agents.

### CROSS REFERENCE TO RELATED APPLICATIONS

This application claims priority based upon U.S. Provisional Patent Application No. 61/638,560, filed Apr. 26, 2012, Provisional Patent Application No. 61/485,091, filed on May 11, 2011, and is a continuation-in-part of U.S. patent application Ser. No. 13/450,094, filed on Apr. 18, 2012. The contents of the foregoing applications are herein incorporated by reference.

### FIELD OF THE INVENTION

This invention dates to Anti-inflammatory treatments and pharmaceutically effective agents therefor.

### BACKGROUND OF THE INVENTION

Diabetes mellitus has become pandemic and according to a forecast by the World Health Organization, there will be a sharp increase in the number of diabetic patients by the year 2030. This is an ominous forecast, because managing the long-term complications of diabetes, which include nephropathy, neuropathy, retinopathy, and cardiovascular complications, will have a serious impact on public health budgets. The hallmark of diabetes is chronically elevated blood glucose levels.

Prediabetes is a syndrome. Many patients with type 2 diabetes and with a prediabetic condition known as metabolic syndrome suffer from a variety of lipid disorders including elevated triglycerides. The body uses triglycerides to store fat but high (>200 mg/dl) and very high (>500 mg/dl) triglycerides are associated with atherosclerosis which increases the patients risk of heart attack and stroke.

Incipient diabetes with impaired glucose tolerance is another prediabetic condition. Overall, type 2 diabetes and incipient diabetes with impaired glucose tolerance, are intimately intertwined with obesity, hyperlipidemia, including hypertriglyceridemia, and cardiovascular complications including arrhythmia, cardiomyopathy, myocardial infarction, stroke and heart failure. Clinically, pre-diabetes means that blood sugar level is higher than normal, but it's not yet increased enough to be classified as type 2 diabetes. Still, without intervention, prediabetes is likely to become type 2 diabetes over time.

It is a matter of great concern that many patients are known to develop resistance to extant antidiabetic agents over time. According to Shoelson et al, in The Journal of Clinical Investigation, 2006, 116, 1793-1801, increased levels of markers and mediators of inflammation correlate with incident type 2 diabetes. Subsequently, A. B. Goldfine et al, in Annals o Internal Medicine, 2010, 152, 346-357 and references cited therein, have shown that the compound of formula I, also known as salsalate, lowers blood glucose, HbA_{1c} levels, triglycerides, free fatty acid and C-reactive protein in patients with type 2 diabetes. Also, salsalate was said to improve glucose utilization and was said to increase circulating insulin. Furthermore, it was said to increase adiponectin concentrations. Unlike any other known antidiabetic, salsalate is thought to target the inflammation component of diabetes. Thus, salsalate may provide a new avenue for treatment.

Very recently, a new aspect of salsalate has come to light. According to Nixon et al, Diabetes (Publish Ahead of Print), published online Feb. 22, 2012, the anti-inflammatory agent salsalate alters glucocorticoid metabolism in mice and humans in a pattern that differs between liver and subcutaneous adipose tissue. Down regulation of intra-adipose 11β-hydroxysteroid dehydrogenase-1 may contribute to the insulin sensitizing effect of salicylates. Also, Shoelson (US 2011/0021468 A1, Jan. 27, 2011) has described the use of salsalate in atherosclerotic cardiovascular disease. Salsalate is thought to be a prodrug of salicylic acid (formula II). Similarly, the compound of the formula IV and the compound of the formula V can also be regarded as prodrugs of salsalate and salicylic acid. Salsalate is a non-steroidal anti-inflammatory agent for oral administration. Salsalate's mode of action as an anti-inflammatory and antirheumatic agent may be due to inhibition of synthesis and release of prostaglandins. The usefulness of salicylic acid, the active in vivo product of salsalate, in the treatment of arthritic disorders has been established. In contrast to aspirin, salsalate causes no greater fecal gastrointestinal blood loss than placebo. The mode of anti-inflammatory action of salsalate and other nonsteroidal anti-inflammatory drugs is not fully defined, but appears to be primarily associated with inhibition of prostaglandin synthesis. This inhibition of prostaglandin synthesis is done through the inactivation of cyclooxygenase-1 (COX-1) and COX-2, which are reponsible for catalyzing the formation of prostaglandins in the arachidonic acid pathway. Salsalate appears to selectively inhibit prostaglandin synthesis in vivo, providing anti-inflammatory activity equivalent to aspirin and indomethacin. Unlike aspirin, salsalate does not inhibit platelet aggregation.

Salsalate (formula II) is thought to be a prodrug of salicylic acid (formula I). Similarly the compound of the formula IV and the compound of the formula V can also be regarded as prodrugs of salsalate and salicylic acid. Consequently, both trisalate and tetrasalate would be expected to share the therapeutic properties, including antidiabetic, anti-atherosclerotic and the GI-sparing anti-inflammatory uses, of salsalate and salicylic acid. However, in contrast to salsalate and salicylic acid, trisalate and tetrasalate have the potential to dial in wider therapeutic doses in convenient dosages. The more water soluble salts of trisalate and tetrasalate would be expected to show higher blood levels to be even more efficacious in animals and humans.

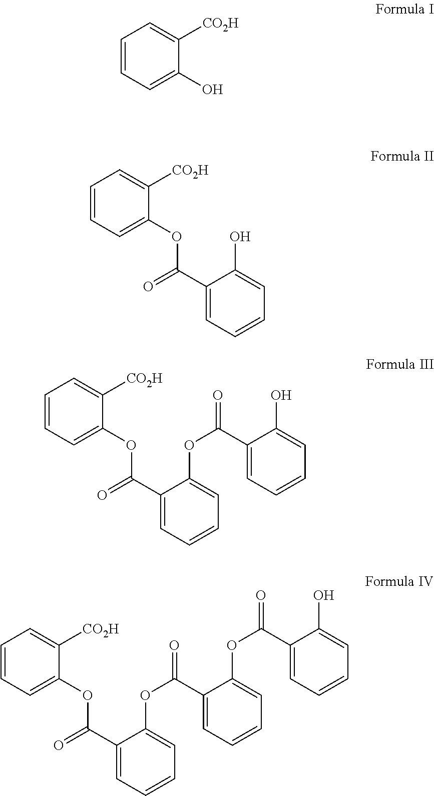 [SMILES: O=C(O)c1ccccc1O ; O=C(Oc1ccccc1C(=O)O)c1ccccc1O ; O=C(Oc1ccccc1C(=O)Oc1ccccc1C(=O)O)c1ccccc1O ; O=C(Oc1ccccc1C(=O)Oc1ccccc1C(=O)Oc1ccccc1C(=O)O)c1ccccc1O]

### SUMMARY OF THE INVENTION

The present invention relates to the use of compounds of formula V and formula VI wherein X is selected from R¹, R², and R³, Y⁺ is a mono-protonated cation of a monobasic amine or a a mono-protonated cation of a dibasic amine and Y⁺⁺ is di-protonated cation of a dibasic amine to treat inflammatory conditions and diseases. The mono-protonated cation of a monobasic amine includes monobasic naturally occurring amino acids such as glycine, alanine, valine, serine, and phenyl alanine. Other monobasic amines include alkyl amines such as methyl amine, ethyl amine and isopropyl amine, or hydroxyalkyl amine such as mono-, di-, tri-ethanol amine or cyclic amines such as glucosamine and morpholine. The dibasic amine includes lysine, arginine and piperazine.

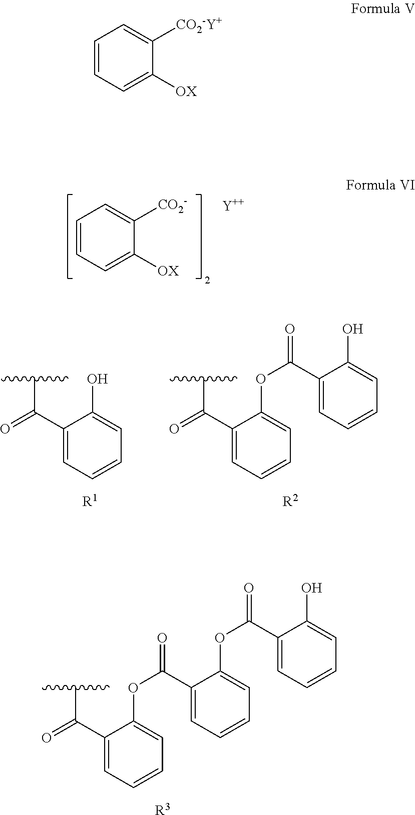

The compounds of the present invention include any polymorphs, solvated, and hydrates of the metformin salts described herein.

In one embodiment of the present invention, the salts of the present invention are the mono-protonated glycine, alanine, valine, and morpholine salt of salicylsalicylic acid, tri-salicylic acid and tetra-salicylic acid. In another embodiment, they may be the mono-protonated arginine, lysine and piperazine salt of salicylsalicylic acid, tri-salicylic acid and tetra-salicylic acid. In another embodiment, they may be the di-protonated arginine, and lysine salt of salicylsalicylic acid, tri-salicylic acid and tetra-salicylic acid. The present invention also relates to a pharmaceutical composition comprising a salt of the present invention and a pharmaceutically acceptable carrier.

In one embodiment, the present invention relates to a pharmaceutical composition for the treatment of diabetes in mammals comprising an anti-diabetes effective amount of a salicylsalicylic, tri-salicylic and tetra-salicylic acid salt of the present invention and a pharmaceutically acceptable carrier. In one embodiment the mammals are humans. The present invention also relates to a method of treating diabetes in a mammal comprising administering to the mammal a salicylsalicylic, tri-salicylic and tetra-salicylic acid salt of the present invention. Preferably, the mammal is a human.

The present invention also relates to a method of treating diabetes in a mammal comprising administering to a mammal in need of said treatment an anti-diabetic effective amount of a salicylsalicylic, tri-salicylic and tetra-salicylic acid salt of the present invention. Preferably, the mammal is a human.

The present invention further relates to a method of treating prediabetes in a mammal comprising administering to a mammal in need of said treatment an anti-prediabetic effective amount of a salicylsalicylic, tri-salicylic and tetra-salicylic acid salt of the present invention. Preferably, the mammal is a human.

The present invention also provides to a method of treating obesity in a mammal comprising administering to a mammal in need of such treatment an antiobesity effective amount of a salicylsalicylic, tri-salicylic and tetra-salicylic acid salt of the present invention. Preferably, the mammal is a human.

The present invention also provides to a method of treating atherosclerotic cardiovascular disease in a mammal comprising administering to a mammal in need of such treatment an anti-atherosclerotic cardiovascular disease effective amount of a salicylsalicylic, tri-salicylic and tetra-salicylic acid salt of the present invention. Preferably, the mammal is a human.

The present invention relates to a method of treating general inflammatory disorders, including rheumatoid arthritis, in a mammal comprising administering to a mammal in need of such treatment an anti-atherosclerotic cardiovascular disease effective amount of a salicylsalicylic, tri-salicylic and tetra-salicylic acid salt of the present invention. In one embodiment of the invention the mammal is a human.

The present invention also relates to a method of treating diabetes, pre-diabetes, obesity, atherosclerotic cardiovascular disease, and general inflammatory disorders, including rheumatoid arthritis, in a mammal comprising administering to a mammal in need of such treatment an anti-atherosclerotic cardiovascular disease effective amount of a salicylsalicylic, tri-salicylic and tetra-salicylic acid salt of the present invention. In one embodiment of the invention the mammal is a human.

One embodiment of the present invention relates to a unit dosage form comprising a salicylsalicylic, tri-salicylic and tetra-salicylic acid salt of this invention.

Another embodiment of the present invention relates to a kit comprising a unit dosage comprising a salicylsalicylic, tri-salicylic and tetra-salicylic acid salt of this invention with instructions on how to use the kit and with provision for at least one container for holding the unit dosage form.

One embodiment of the present invention relates to a unit dosage form comprising a salicylsalicylic, tri-salicylic and tetra-salicylic acid this invention.

One embodiment of the present invention relates to a kit comprising a unit dosage comprising a salicylsalicylic, tri-salicylic and tetra-salicylic acid of this invention with instructions on how to use the kit and with provision for at least one container for holding the unit dosage form.

The terms “treating”, “treat”, or “treatment” as used herein include curative, preventive (e.g., prophylactic) and palliative treatment.

### DETAILED DESCRIPTION OF THE INVENTION

The compounds of the present invention, i.e., the salts of salicylsalicylic acid, tri-salicylic acid and tetra-salicylic acid with monobasic amines and dibasic amines can be prepared as set forth below.

One equivalent of a monobasic amine, may be dissolved in an appropriate reaction inert solvent. The solvent may be a polar solvent such as water. As used herein, the expression “reaction inert solvent” refers to a solvent or a mixture of solvents which does not interact with starting materials, reagents, intermediates or products in a manner which adversely affects the yield of the desired product. Preferred solvents include methanol, ethanol, n-propanol, sopropanol, acetonitrile, acetone, ethyl methyl ketone, diethyl ketone and methyl isobutyl ketone. Particularly preferred solvents for this reaction are acetonitrile, acetone and methyl isobutyl ketone. To this solution may be added a solution of one equivalent of salicylic acid, also known as salsalate and (2-(2-hydroxybenzoyl)oxybenzoic acid), trisalicylic acid or tetrasalicylic acid in a reaction inert solvent (Salsalate is commercially available.) The reaction mixture can be stirred at about ambient temperature to about the reflux temperature of the solvent being used for about two hours to about six hours, preferably at ambient temperature for about two hours. The compounds of this invention can be isolated from the reaction mixture by methods well known to those skilled in the art, including according to the method of U.S. Pat. No. 3,957,853.

Trisalicylic acid is described by Don Carlos Monserrat Vidal et al (ES 8308897 A1, “Procedimiento Para Obtencion De Polimeros Del Acido 2-Hydroxibenzoico”) and tetrasalicylic acid is described by O. Shulga and J. Dunn (U. 2004, 410, 15-21). Furthermore, trisalicylic acid and tetrasalicylic acid can be prepared by adapting procedures described by Galzigna, L. et al. in Farmaco, 1993, 48(1), 95-103 with the title “Synthesis and some properties of two salsalate derivatives.” Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

### Oral Administration

The compounds of the invention may be administered orally. Formulations suitable for oral administration include solid formulations, such as tablets, capsules Containing particulates, liquids, or powders; lozenges (including liquid-filled), chews; multi- and nano-particulates; gels, solid solution, liposome, films (including muco-adhesive), ovules, sprays, and liquid formulations.

### Dosage

For administration to human patients, the total daily dose of the compounds of the invention is typically in the range 1 g to 12 g depending, of course, on the mode of administration. In one embodiment, the total daily dose is in the range 1 g to 10 g and in another embodiment, the total daily dose is in the range 4 g to 8 g. The total daily dose may be administered in single or divided doses. These dosages are based on an average human subject having a weight of about 65 kg to 70 kg. The physician will readily be able to determine doses for subjects whose weight falls outside this range, such as infants and the elderly.

The pharmaceutical composition may, for example, be in a form suitable for oral administration as a tablet, capsule, pill, powder, sustained release formulations, solution, or suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier and a compound according to the invention as an active ingredient. In addition, it may include other medicinal or pharmaceutical agents, carriers, adjuvants, etc.

Suitable pharmaceutical carriers include inert diluents or fillers, water and various organic solvents. The pharmaceutical compositions may, if desired, contain additional ingredients such as flavorings and binders. Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Compounds of the formula II, III, IV, V, and VI can be tested for anti-diabetes activity as follows. Male Wistar rats 8-10 weeks of age 210-230 g. of body weight (bw) are used. The rats are housed at temperature of 18-21° C. on a 12 hour light-dark cycle. Rats are fed on a stock laboratory diet (59% carbohydrates, 17% protein, 3% fat, 21% minerals, water, and cellulose) and are allowed water ad libitum. Diabetes mellitus is induced in Wistar male rats by two intravenous injections of alloxan (40 mg/kg bw) in the tail vein. The rats are used in experiments 6 days after the first alloxan injection. Fasting glucose, insulin, total cholesterol, and triglycerides levels of these animals are recorded. Then rats are treated with metformin hydrochloride (100-300 mg/kg bw) for the next 5 days. On the sixth day. Fasting glucose, insulin, total cholesterol, and triglycerides levels of these animals are recorded.

Compounds of the formula II, III, IV, V and VI can also be tested for anti-diabetes activity as follows. Spontaneously diabetic Bio-Bred (BB/W) rats from the colony maintained at the University of Massachusetts Medical Center, Worcester, were used in this study. BB/W rats were chosen for the current study because the BB/W rats have been considered a useful model of autoimmune human insulin-dependent diabetes DM). Like human IDDM, spontaneous diabetes appears during adolescence, with an abrupt clinical onset characterized by weight loss, hyperglycemia, hypoinsulinemia, and ketonuria. As in the case of human diabetics, pathological changes in retina, myocardium, liver, kidney, bone metabolism and peripheral nerves have all been well documented in BB rats, as described in Diab. Metab. Rev., 8:9 (1992). The BB/W rats were 3 to 4 months old and weighed about 300 to 350 g. The BB/W rats received daily insulin, which was discontinued 24 h prior to performing the isolated heart perfusion studies, leading to a hyperglycemic state. The rats were acutely diabetic, receiving 2.02±0.04 units of insulin daily, and had been diabetic for at least 12±3 days. The mean blood glucose levels in these diabetic rats were 386±24 mg/dL. The age-matched non-diabetic controls had mean blood glucose levels of 92±12 g/dL.

Animal models to determine the effects of compounds of the invention on diabetes and complications of diabetes have been reviewed by Tirabassi et al., ILAR Journal, 2004, 45, 292-302. Antidiabetic activity may also be tested according to protocols described in the following patents: U.S. Pat. Nos. 4,340,605; 4,342,771; 4,367,234; 4,617,312; 4,687,777 and 4,703,052. Additional references relevant to this application include the following: French Patent 2796551 and United States Published Patent Application No. 20030220301.

Efficacy in treating prediabetes can be determined according to protocols described by Armato et al in Endocrinology Practice, 2011, Nov. 8: 1-21 (Epub ahead of print).

Efficacy in ameliorating atherosclerotic cardiovascular disease can be determined according to Shoelson et al (US 2011/0021468 A1, Jan. 27, 2011).

One mole of monobasic amine is dissolved in 3 L of acetone contained in a concentration reactor. To this solution, one mole of salicylsalicylic acid dissolved in 1 L of acetone is added and the reaction is stirred for about one to two hours. Excess acetone is cautiously evaporated under vacuum at or below room temperature. The resulting solid is collected and dried to obtain the monobasic amine mono-salt of salicylsalicylic acid.

One mole of dibasic amine is dissolved in 3 L of acetone contained in a concentration reactor. To this solution, one mole of salicylsalicylic acid dissolved in 1 L of acetone is added and the reaction is stirred for about one to two hours. Excess acetone is cautiously evaporated under vacuum at or below room temperature. The resulting solid is collected and dried to obtain the dibasic amine mono-salt of salicylsalicylic acid.

One mole of dibasic amine is dissolved in 3 L of acetone contained in a concentration reactor. To this solution, two moles of salicylsalicylic acid dissolved in 1 L of acetone is added and the reaction is stirred for about one to two hours. Excess acetone is cautiously evaporated under vacuum at or below room temperature. The resulting solid is collected and dried to obtain the dibasic amine di-salt of salicylsalicylic acid.

### Rat Paw Edema Anti-Inflammatory Assay

Adult male Wistar rats (100-200 g) are used throughout this study. Fifteen rats, five per cage, are housed in a room maintained at a constant temperature of 24-26° C. with 12-h light/dark cycle and had free access to food and water. Before experimentation, animals have 1 week to adapt to the conditions of the facility. Prior to the start of the experiment, body weights are measured individually to determine proper treatment dose, and animals are randomly divided into three different groups of five rats. Initial paw sizes (basal volume) are measured by a volume displacement method using a digital plethysmometer (Ugo Basile, Comerio Va., Italy).

Paw edemas are induced by subcutaneous injection of 100 μl of 1% lambda carrageenan solution (Sigma) (w/v solution in saline, 0.9% NaCl) in the plantar aponeurosis of the right hind paw. Carrageenan is a sulfated polysaccharide that promotes acute inflammation by activating proinflammatory cells. One hour after carrageenan injection, PEO and test compounds, for example, trisalate are orally gavaged. An equal volume of the vehicle (4% apricot kernel balm) is given to the control group. Edemas are measured 3, 5, 24, and 48 h after PEO and test compound treatments. Time-dependent paw edema size reduction reflected the anti-inflammatory effect of the specific treatment. The increase in volume and the percent change caused by the irritant are estimated after subtracting the basal volume of the paw before injection. A lower numeric value (in percent) would indicate stronger anti-inflammatory activity.

1. A composition of matter comprising compounds of the formula: 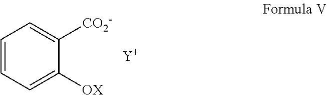
and compounds of the formula 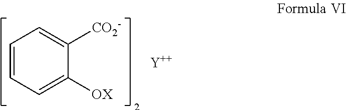
wherein X is R¹, R², or R³, wherein
R¹ is 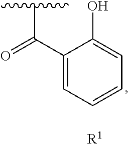
and R² is 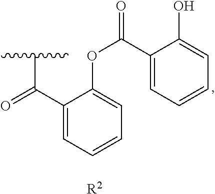
and R³ is 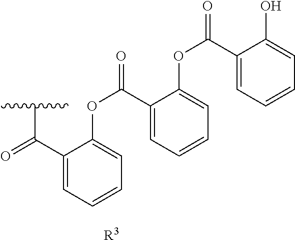
and Y⁺ is a mono-protonated cation of a monobasic amine or a a mono-protonated cation of a dibasic amine and Y⁺⁺ is di-protonated cation of a dibasic amine, wherein the mono-protonated cation of a monobasic amine includes a monobasic naturally occurring amino acids including glycine, alanine, valine, serine, phenyl alanine, an alkyl amines including methyl amine, ethyl amine and isopropyl amine, or a hydroxyalkyl amine including mono, di-, tri-ethanol amine or cyclic amines such as glucosamine and morpholine, and wherein the dibasic amine includes lysine, arginine and piperazine.

2. A pharmaceutical composition comprising a compound of the formula: 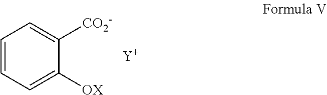
wherein Y⁺ is H, and X is R¹, R², or R³, and wherein
R¹ is 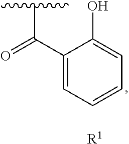
and R² is 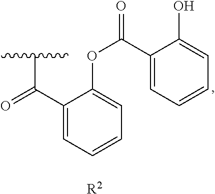
and R³ is 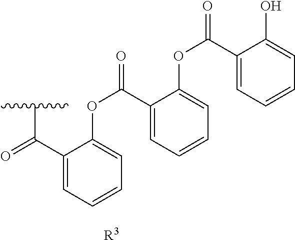
and a pharmaceutically acceptable carrier.

3. A pharmaceutical composition comprising a compound according to claim 1 and a pharmaceutically acceptable carrier.

4. A pharmaceutical composition according to claim 1 wherein said compound is present in an anti-inflammatory effective amount.

5. A kit comprising a) a unit dosage comprising the compound of claim 1; b) instructions on how to use the kit; and c) at least one container for holding the unit dosage forms.

6. A method of treating an inflammatory disease or condition in a mammal, including a human, comprising administering to the mammal in need of such treatment a compound according to claim 1.

7. A method of treating inflammation in a mammal, including a human, comprising administering to the mammal in need of such treatment an anti-inflammatory effective amount of a compound according to claim 1.

8. A method of treating pre-diabetes in a mammal, including a human, comprising administering to the mammal in need of such treatment a pre-antidiabetic effective amount of a compound according to claim 1.

9. A method of treating obesity in a mammal, including a human, comprising administering to the mammal in need of such treatment an anti-obesity effective amount of a compound according to claim 1.

10. A method of treating or preventing the development or progression of atherosclerotic cardiovascular disease in a mammal, including a human, comprising administering to the mammal an effective amount of the compound according to claim 1.

11. A method for the manufacture of the composition of claim 1 comprising fusing the amine with salicylsalicylic acid, trisalicylic acid or tetrasalicylic acid in a reaction inert solvent at a temperature between about 0 degrees C. and about 60 degrees C.

12. A method for the manufacture of the composition of claim 1 comprising reacting the amine with salicylsalicylic acid, trisalicylic acid or tetrasalicylic acid at a temperature between about 50 degrees C. and about 200 degrees C.

13. A pharmaceutical composition comprising a compound according to claim 2 and a pharmaceutically acceptable carrier.

14. A pharmaceutical composition according to claim 2 wherein said compound is present in an anti-inflammatory effective amount.

15. A kit comprising a) a unit dosage comprising the compound of claim 2; b) instructions on how to use the kit; and c) at least one container for holding the unit dosage forms.

16. A method of treating an inflammatory disease or condition in a mammal, including a human, comprising administering to the mammal in need of such treatment a compound according to claim 2.

17. A method of treating inflammation in a mammal, including a human, comprising administering to the mammal in need of such treatment an anti-inflammatory effective amount of a compound according to claim 2.

18. A method of treating pre-diabetes in a mammal, including a human, comprising administering to the mammal in need of said treatment a pre-antidiabetic effective amount of a compound according to claim 2.

19. A method of treating obesity in a mammal, including a human, comprising administering to the mammal in need of such treatment an anti-obesity effective amount of a compound according to claim 2.

20. A method of treating or preventing the development or progression of atherosclerotic cardiovascular disease in a mammal, including a human, comprising administering to the mammal an effective amount of the compound according to claim 2.
